(19) United States
(12) Patent Application Publication
(10) Pub. No.: US 2007/0260321 A1
(43) Pub. Date: Nov. 8, 2007

(54) Conically-shaped glenoid implant with a prosthetic glenoid insert used in total shoulder arthroplasty and method

(76) Inventor: Robert P. Stchur, Punta Gorda, FL (US)

Correspondence Address: EDWARD M. LIVINGSTON, PA, 963 TRAIL TERRACE DRIVE, NAPLES, FL, 34103, US

(21) Appl. No.: 11/415,594

(22) Filed: May 2, 2006

(51) Int. Cl. A61F 2/40

(52) U.S. Cl. 623/19.11

(57) ABSTRACT

A conically-shaped glenoid implant (1) having a fastening means for the acceptance of a conventional prosthetic glenoid insert (7) and a hole (9) located thereon for the acceptance of a locking means. The fastening means are preferably notches (11) while the locking means is preferably reverse barbs (13). An optional screw (3) or keels (19) may be located on the exterior surface of the cone (2) for additional securement of the conically-shaped glenoid implant (1) in the glenoid (6) and to prevent rotation of the cone (27). By using the present invention, bone ingrowth is achieved because sheer forces are converted to compressive forces and surface area for ingrowth is significantly increased.

### BACKGROUND OF THE INVENTION

This invention relates to prosthetic glenoid components, more particularly, a conically-shaped glenoid implant for holding a prosthetic glenoid insert used in total shoulder arthroplasty wherein the device is conical in shape to provide a larger amount of surface area and to convert sheer force into compressive force. A special tool is also provided to assist in the process.

Arthritis or other injuries to the shoulder typically cause a patient to seek cures for shoulder stiffness and pain. Although not required, total shoulder arthroplasty is often the most effective method for restoring movement and comfort to shoulders damaged by arthritis, injury or other surgeries.

In total shoulder arthroplasty, the surfaces of the humeral head of the humerus (commonly referred to as “the ball”) and the glenoid (commonly referred to as “the socket”) are resurfaced with metal and plastic implants, thereby restoring movement and comfort to the patient. The humeral prosthesis is usually press fit into the humerus; alternatively, it may be cemented into the shaft of the humerus. Then, the glenoid bone shape and orientation are corrected using a circular reamer and a polyethylene glenoid insert is inserted into the glenoid. A small amount of bone cement is used to hold the polyethylene glenoid insert in place. Thus, the prosthetic humeral head and glenoid are now correctly aligned so as to provide maximum movement without pain.

However, although the results obtained from undergoing total shoulder arthroplasty are initially positive, the prosthetic polyethylene glenoid insert does not remain in a permanent position due to glenoid fixation, thus necessitating additional surgeries to replace the prosthetic polyethylene glenoid insert. The glenoid fixation problem is, in large part, due to the joint being in constant sheer due to the upward and downward movement of the deltoid, thus being non-conducive to bone ingrowth. Although there have been many attempts to stabilize the prosthetic glenoid using keels, screws, etc., the sheer overcomes these stabilizing means, thereby eliminating the possibility of bone ingrowth. In addition, the secondary “rocker horse” eccentric loads placed on the glenoid creates an environment that is not conducive to bone ingrowth.

Furthermore, previous glenoid designs have minimal bone-prosthetic interface surface area to allow for adequate bone ingrowth. Thus, the need for bone ingrowth is of great importance in achieving total shoulder arthroplasties that have long-lasting, positive results.

Although sheer force is not conducive to bone ingrowth, compressive force is conducive to bone ingrowth. Thus, rather than having sheer force acting upon the shoulder, it would be most beneficial to have compressive forces acting on the glenoid in order to promote boney ingrowth.

Thus, a need exists for a conical prosthetic glenoid device and method that converts sheer force into compressive force and also increases the surface area at the bone-prosthetic interface to promote bone ingrowth.

The relevant prior art includes the following references:

| Patent No. | | |
|---|---|---|
| (U.S. unless stated otherwise) | Inventor | Issue/Publication Date |
| 2006/0009852 | Winslow et al. | Jan. 12, 2006 |
| 5,080,673 | Burkhead et al. | Jan. 14, 1992 |
| 6,699,289 | Iannotti et al. | Mar. 2, 2004 |
| 3,916,451 | Buechel et al. | Nov. 4, 1975 |
| 5,723,018 | Cyprien et al. | Mar. 3, 1998 |
| 6,790,234 | Frankle | Sep. 14, 2004 |
| 6,146,425 | Hoermansdoerfer | Nov. 14, 2000 |
| 4,693,723 | Gabard | Sep. 15, 1987 |
| 2005/0113931 | Horber | May 26, 2005 |
| JP404300539A | Taeger | Oct. 23, 1992 |

### SUMMARY OF THE INVENTION

The primary object of the present invention is to provide a conically-shaped glenoid implant and method that converts sheer force to compressive force.

A further object of the present invention is to provide a conically-shaped glenoid implant and method that promotes bone ingrowth.

An even further object of the present invention is to provide a conically-shaped glenoid implant and method that has a large surface area for boney ingrowth to occur.

Another object of the present invention is to provide a conically-shaped glenoid implant and method that can remain in the glenoid for a long length of time.

The present invention fulfills the above and other objects by providing a conically-shaped glenoid implant having a fastening means for the acceptance of a conventional prosthetic glenoid insert and a hole located thereon for the acceptance of a locking means. The fastening means are preferably notches while the locking means is reverse barbs. An optional cannulated screw or keels may be located on the exterior surface of the cone for additional securement of the conically-shaped glenoid implant and to prevent rotation of the conically-shaped glenoid implant in the glenoid. The conically-shaped glenoid implant is preferably coated and porous so as to permit boney ingrowth to occur.

To install the conically-shaped glenoid implant of the present invention, a surgeon first drills a central guide wire into the glenoid. Then, using a cannulated drill, he or she begins drilling a hole in the glenoid. The hole is then reamed using progressively larger reamers so as to create a conical hole smaller than the prosthesis. A cone punch is then used to perfect the shape of the hole into a conically-shaped hole that is slightly smaller than the device. A cannulated inserter is then inserted through the screw hole of the conically-shaped glenoid implant and into the glenoid itself. The conically-shaped glenoid implant is then tapped into place in the glenoid to achieve a press fit. The use of reverse barbs may also assist in retaining the implant in the glenoid. Further locking means, preferably a locking screw, is threaded through the screw hole of the conically-shaped glenoid implant. Finally, the guide wire is removed and a conventional prosthetic glenoid insert is impacted onto the fastening means, which are preferably notches, although current locking mechanism are also acceptable.

The above and other objects, features and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

In the following detailed description, reference will be made to the attached drawings in which:

FIG. 1 is an anterior plan view of the conically-shaped glenoid implant of the present invention installed in a right glenoid;

FIG. 2 is a perspective frontal view of the conically-shaped glenoid implant of the present invention;

FIG. 3 is a perspective side view of the conically-shaped glenoid implant of the present invention;

FIG. 4 is a frontal view of the face of the conically-shaped glenoid implant of the present invention;

FIG. 5 is a perspective frontal view of a second embodiment of the conically-shaped glenoid implant of the present invention;

FIG. 6 is a side view of a third embodiment of the conical conically-shaped glenoid implant of the present invention;

FIG. 7 is a frontal view of the face of the third embodiment of the conically-shaped glenoid implant of the present invention;

FIG. 8 is a perspective view of a first step of installing a conically-shaped glenoid implant of the present invention in a glenoid;

FIG. 9 is a perspective view of a second step of installing a conically-shaped glenoid implant of the present invention in a glenoid;

FIG. 10 is a perspective view of a third step of installing a conically-shaped glenoid implant of the present invention in a glenoid;

FIG. 11 is a perspective view of a fourth step of installing a conically-shaped glenoid implant of the present invention in a glenoid;

FIG. 12 is a perspective view of a fifth step of installing a conically-shaped glenoid implant of the present invention in a glenoid;

FIG. 13 is a perspective view of a sixth step of installing a conically-shaped glenoid implant of the present invention in a glenoid;

FIG. 14 is a perspective view of a seventh step of installing a conically-shaped glenoid implant of the present invention in a glenoid; and

FIG. 15 is a perspective view showing the forces placed upon the conically-shaped glenoid implant of the present invention.

### DESCRIPTION OF THE PREFERRED EMBODIMENTS

For purposes of describing the preferred embodiment, the terminology used in reference to the numbered components in the drawings is as follows:

1. conically-shaped glenoid

2. cone exterior surface

3. screw

4. humeral head

5. humerus

6. glenoid

7. prosthetic glenoid insert

8. coneapex

9. hole for fastening means

10. cone base

11. notches

12. cone interior surface

13. barb

14. cannulated drill

15. site for insertion of conically-shaped implant, generally glenoid implant

16. center of glenoid

17. reamer

18. cone punch

19. keel

20. cannulated inserter

21. height of cone

22. width of cone base

23. width of cone apex

24. retractor

25. central guide wire

26. deltoid muscle

27. cone

28. compressive force

29. sheer force

Referring to FIG. 1 and other applicable drawings, currently, the total shoulder arthroplasty procedure includes replacing the humeral head 4 and glenoid 6 with prosthetic devices that provide for the proper articulation of the humeral head 4 and the glenoid 6. First, the surgeon makes an incision across the front of the shoulder from the coracoid process to the middle of the humerus 5. Then, the muscles and other tissues near the shoulder are mobilized and the humerus 5 is cut and prepared for the prosthesis. Then, the surgeon corrects the shape and orientation of the glenoid 6 using a reamer 17. Finally, a prosthetic glenoid insert 7, which is preferably polyethylene in nature but may include other materials, is inserted into the glenoid 6. Bone cement is used to secure the prosthetic glenoid insert 7 to the glenoid 6 and the subscapularis tendon is fixed to the humerus 2.

Although the prosthetic glenoid insert 7 is installed in the glenoid 6, most likely the patient will require another surgery to replace the prosthetic glenoid insert 7 due to up and down movement of the deltoid muscle, also known as sheer force 29. This sheer force 29 eventually leads to loosening at the bone-cement interface and, ultimately, failure of the glenoid implant. In previous attempts at ingrowth, glenoid designs have failed to overcome this sheer force 29, thereby preventing the prosthetic glenoid insert 7 from achieving bone ingrowth within the glenoid and failing to become a permanent fixture in the glenoid 6.

On the other hand, the use of the conically-shaped glenoid implant 1 of the present invention in conjunction with the prosthetic glenoid insert 7 does promote bone ingrowth as the conically-shaped glenoid implant 1 includes a porous coat. In addition, because the surface area of the conically-shaped glenoid implant 1 is markedly larger than the surface area of the ovular prosthetic glenoid insert 7, the likelihood of achieving bone ingrowth is much greater. For example, a conically-shaped glenoid implant 1 with a 28 millimeter diameter would have a surface area of 1,457 mm² as determined by the formula for surface area of a cone being IIrs where s equals slant height and slant height is the square root of r²+h². On the other hand, a standard ovular prosthetic glenoid insert 7 having a size of 40 mm by 28 mm would have a surface area of only 879 mm² as determined by the formula II(L×W/2). Therefore, because the conically-shaped glenoid implant 1 has a larger surface area when compared to the prosthetic glenoid insert 7, the chances of bone ingrowth occurring are greater. Thus, the conically-shaped glenoid implant 1 would, at least theoretically, last forever in the glenoid 6 without the need for additional surgeries.

Specifically focusing on the conically-shaped glenoid implant 1 of the present invention, with reference to FIG. 1, an anterior plan view of the conically-shaped glenoid implant 1 of the present invention installed in a right glenoid is shown. The conically-shaped glenoid implant 1 of the present invention is installed in a glenoid 6, which is also known as a shoulder socket, to provide long-lasting securement of a prosthetic glenoid insert 7, which may be made of polyethylene or any other material, within the glenoid 6. Once inserted, the prosthetic glenoid insert 7 provides a smooth, slightly concave surface for articulation with the humeral head 4 located on the proximal end of the humerus 5. The conically-shaped glenoid implant 1 is inserted into the glenoid 6 and is secured therein via a fastening means, such as a screw 3, or preferably reverse barbs shown in FIGS. 6 and 7, to provide compression and stabilization of the conically-shaped glenoid implant 1 in the glenoid 6.

FIG. 2 shows a perspective frontal view of the conically-shaped glenoid implant of the present invention. The conically-shaped glenoid implant 1 is a hollow cone 27 of a predetermined size that is preferably porous in nature and includes a cone exterior surface 2 and a cone interior surface 12 that is predeterminedly sized and shaped for the acceptance of a prosthetic glenoid insert 7. The conically-shaped glenoid implant 1 has a cone base 10 and a cone apex 8. Located on the cone base 10 is at least one locking means, which is preferably two notches 11, for the acceptance and locking of a prosthetic glenoid insert 7. Located on the cone apex 8 is a hole for an optional fastening means 9 in order to better secure the conically-shaped glenoid implant 1 within the glenoid 6.

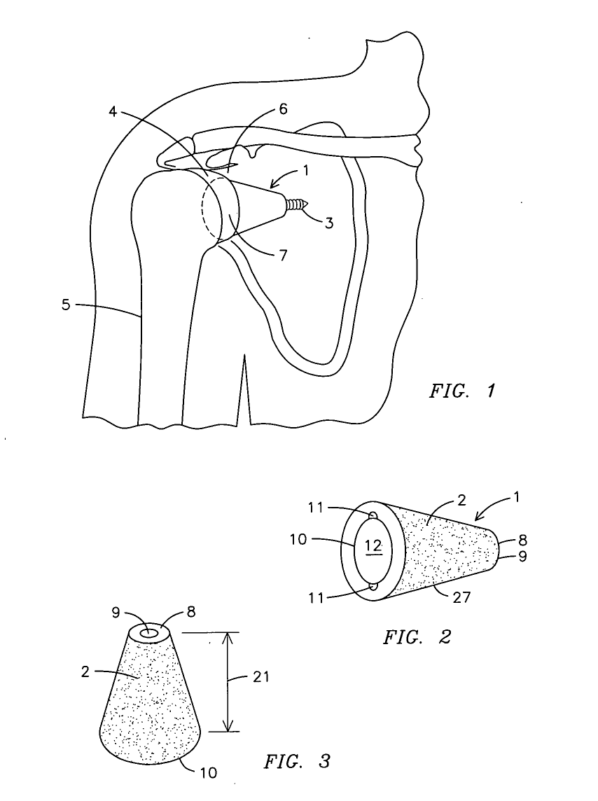

In FIG. 3, a perspective side view of the prosthetic glenoid device of the present invention is shown. The conically-shaped glenoid implant 1 is circular at all planes perpendicular to its axis along the entire height of the cone 21.

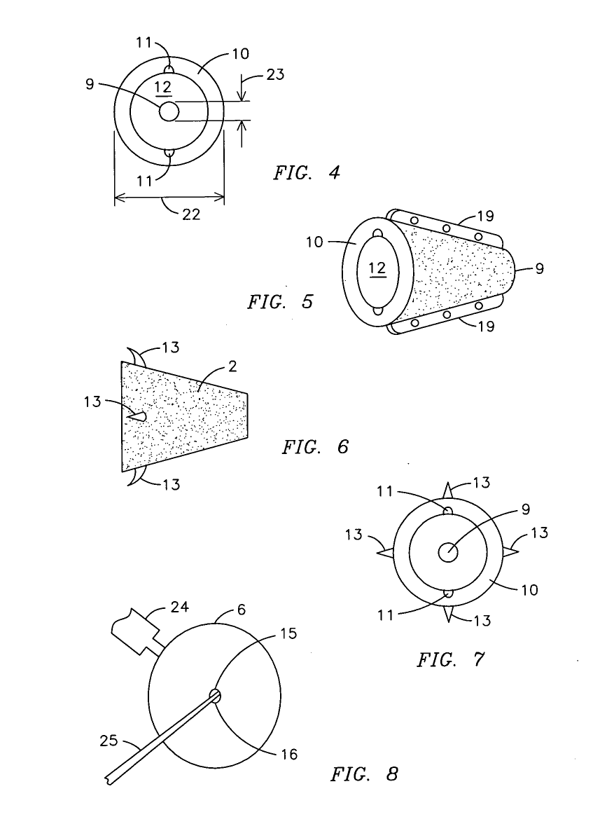

With reference to FIG. 4, a frontal view of the face of the conically-shaped glenoid implant of the present invention is shown. The cone base 10 has a predetermined width 22 and the cone apex 8 has a predetermined width 23 sized smaller than the predetermined width 22 of the cone base 10.

Next, FIG. 5 is a perspective frontal view of a second embodiment of the conically-shaped glenoid implant of the present invention. The second embodiment of the conical conically-shaped glenoid implant 1 includes at least one keel 19 that aids in the prevention of rotation of the conically-shaped glenoid implant 1 within the glenoid 6.

In FIGS. 6 and 7, varying views of a third embodiment of the prosthetic glenoid device of the present invention is shown. At least one reverse barb 13 may be located on the cone exterior surface 2 so as to aid in locking and holding the conically-shaped glenoid implant 1 within the glenoid 6. Although reverse barbs 13 are shown, other projections of various sizes and shapes may be located on the cone exterior surface 2 to aid in locking and holding the conically-shaped glenoid implant 1 within the glenoid 6.

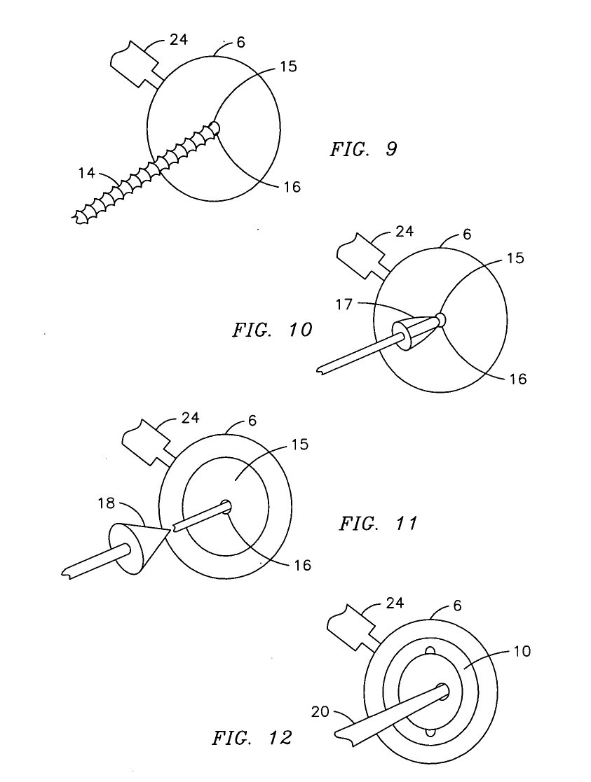

With regards to FIGS. 8 through 14, perspective views of the steps of the method for installing a conically-shaped glenoid implant of the present invention in a glenoid is shown. First, the surgeon positions the retractor 24 such that it is posterior to the glenoid 6. Then, a central guide wire 25 is drilled into a center of the glenoid 16 that is centrally located in a site for insertion of the conically-shaped glenoid implant 15.

A cannulated drill 14 is then positioned on the center of the glenoid 16 in order to begin drilling a hole for the location for placement of the conically-shaped glenoid implant 15. Progressively larger cone reamers 17 are then used to enlarge the site for insertion of the conically-shaped glenoid implant 15 so as to be sized slightly smaller in diameter than the diameter of the conically-shaped glenoid implant 1. It is most advantageous to use the reamers 17 to enlarge the site for insertion of the conically-shaped glenoid implant 15 as it is a quicker and cleaner alternative for shaping and sizing the site 15. However, the surgeon should stop using the reamers 17 as soon as he or she is near a predetermined site size slightly smaller than the conically-shaped glenoid implant 1. Reaming should be carried out to the anterior and posterior inside edge of the cortical glenoid vault.

Next, a cone punch 18 may be used to punch a shape and size in the glenoid 6 slightly smaller than the size of the conically-shaped glenoid implant 1. By using the cone punch 18, a surgeon can shape the exact size of the site 15 if desired.

A cannulated inserter 20 is then inserted into the hole for fastening means 9 in the conically-shaped glenoid implant 1 and is used for the insertion of the conically-shaped glenoid implant 1 into the site for insertion 15. The conically-shaped glenoid implant 1 is then tapped into place within the glenoid 6. A fastening means, such as a cannulated screw 3, is then secured within the hole for fastening means 9 in the conically-shaped glenoid implant 1 to further aid in the securement of the conically-shaped glenoid implant 1 within the glenoid 6.

Finally, the guide wire 25 is removed and the polyethylene glenoid insert 7 is impacted or snapped onto the conically-shaped glenoid implant 1 via the locking means, which is preferably notches 11. It is important to realize that while the embodiment shown here is on a cannulated system for easier installation, its function and efficacy do not rely on the cannulation and is not necessary.

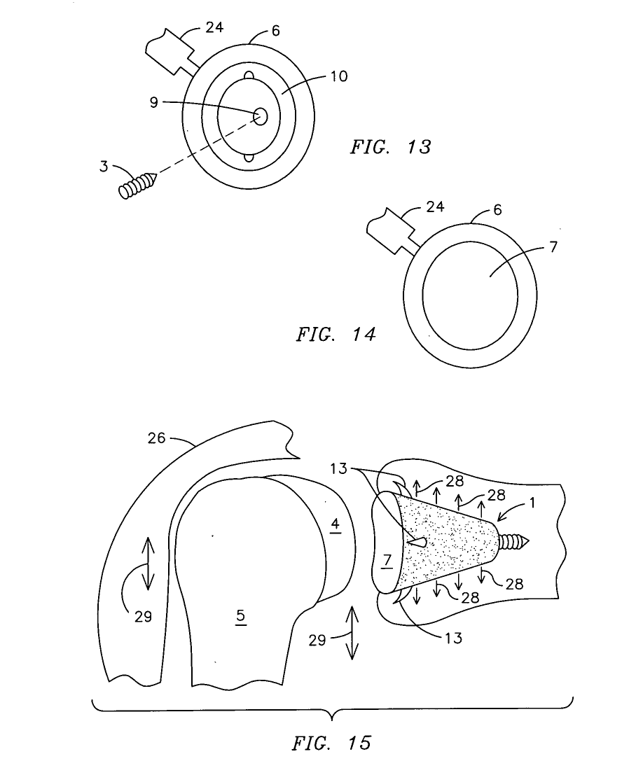

With respect to FIG. 15, upward and downward sheer forces 29 placed upon the conically-shaped glenoid implant 1 of the present invention are shown. When the deltoid muscle 26 is moved, upward and downward sheer force 29 is placed on the glenoid 6. When using a prosthetic glenoid insert 7 alone, the sheer force 29 rotates the prosthetic glenoid insert 7, thereby making it virtually impossible to achieve bone ingrowth. When the conically-shaped glenoid implant 1 is installed in a glenoid and the prosthetic glenoid insert 7 is secured therein, the sheer forces 29 are converted to compressive forces 28 on the conically-shaped glenoid implant 1, thereby aiding in bone ingrowth.

In addition, because the prosthetic device of the present invention is conically-shaped, a surgeon may use both a retractor 24 and a cannulated reamer 17 to ream a location for the conically-shaped glenoid implant 15 that is less than the width of the glenoid 6. Without the use of the present invention, it was difficult for a surgeon to use both instruments at once as the retractors blocked the use of the reamers.

Finally, because the glenoid cortical vault is preserved, a surgeon may revisit the glenoid 6 in the future if necessary to revise the glenoid 6.

The use of the present invention will convert sheer force to compressive force and greatly increase the prosthetic-bone surface area interface, thereby aiding in the promotion of bone ingrowth.

It is to be understood that while a preferred embodiment of the invention is illustrated, it is not to be limited to the specific form or arrangement of parts herein described and shown. It will be apparent to those skilled in the art that various changes may be made without departing from the scope of the invention and the invention is not be considered limited to what is shown and described in the specification and drawings.

Having thus described my invention, I claim:

1. A conically-shaped glenoid implant for installation in a glenoid comprising:
a predeterminedly-sized cone having an interior and exterior surface;
said cone having a base and an apex;
a hole for a fastening means located on said cone apex;
a fastening means for fastening said cone in a glenoid;
a prosthetic glenoid insert; and
a means for locking the prosthetic glenoid insert in said cone.

2. The conically-shaped glenoid implant of claim 1 wherein:
the prosthetic glenoid insert is made of polyethylene.

3. The conically-shaped glenoid implant of claim 1 wherein:
said fastening means for fastening said cone in a glenoid is a screw.

4. The conically-shaped glenoid implant of claim 2 wherein:
said fastening means for fastening said cone in a glenoid is a screw.

5. The conically-shaped glenoid implant of claim 1 wherein:
said means for locking a prosthetic glenoid insert in said cone is at least one notch located on said cone base.

6. The conically-shaped glenoid implant of claim 4 wherein:
said means for locking a prosthetic glenoid insert in said cone is at least one notch located on said cone base.

7. The conically-shaped glenoid implant of claim 1 wherein:
said cone exterior surface is porous.

8. The conically-shaped glenoid implant of claim 1 wherein:
said cone interior surface is predeterminedly sized and shaped for the acceptance of a prosthetic glenoid insert.

9. The conically-shaped glenoid implant of claim 6 wherein:
said cone interior surface is predeterminedly sized and shaped for the acceptance of a prosthetic glenoid insert.

10. The conically-shaped glenoid implant of claim 1 further comprising:
at least one keel located on said cone exterior surface.

11. The conically-shaped glenoid implant of claim 1 further comprising:
at least one barb located on said cone exterior surface.

12. The conically-shaped glenoid implant of claim 9 further comprising:
at least one keel located on said cone exterior surface.

13. The conically-shaped glenoid implant of claim 9 further comprising:
at least one barb located on said cone exterior surface.

14. The conically-shaped glenoid implant of claim 10 further comprising:
at least one barb located on said cone exterior surface.

15. A conically-shaped glenoid implant for installation in a glenoid comprising:
a predeterminedly-sized cone having an interior and exterior surface;
said cone having a base and an apex;
a hole for a fastening means located on said cone apex;
a fastening means for fastening said cone in a glenoid;
said fastening means for fastening said cone in a glenoid is a screw;
a means for locking a prosthetic glenoid insert in said cone;
said means for locking a prosthetic glenoid insert in said cone is at least one notch located on said cone base;
said cone interior surface is predeterminedly sized and shaped for the acceptance of a prosthetic glenoid insert;
a prosthetic glenoid insert; and
said cone exterior surface is porous.

16. The conically-shaped glenoid implant of claim 15 further comprising:
at least one keel located on said cone exterior surface.

17. The conically-shaped glenoid implant of claim 15 further comprising:
at least one barb located on said cone exterior surface.

18. A method for using a conically-shaped glenoid implant for installation in a glenoid comprising a predeterminedly-sized cone having an interior and exterior surface; said cone having a base and an apex; hole for a fastening means located on said cone apex; a fastening means for fastening said cone in a glenoid; a prosthetic glenoid insert; and a means for locking a prosthetic glenoid insert in said cone, said method comprising the steps of:
a. drilling a hole in said glenoid;
b. inserting said cone in said hole; and
c. inserting said prosthetic glenoid insert in said cone.

19. The method of claim 18 further comprising the steps prior to step c of:
d. inserting a screw in said hole for fastening means; and
e. tightening said screw into said glenoid.

20. The method of claim 19 further comprising a step prior to step e of:
f. locking said prosthetic glenoid insert in said cone.
